(19) United States
(12) Patent Application Publication
(10) Pub. No.: US 2010/0198029 A1
(43) Pub. Date: Aug. 5, 2010

(54) Patient Monitoring Using Combination of Continuous Wave Spectrophotometry and Phase Modulation Spectrophotometry

(75) Inventor: Shih-Ping WANG, Los Altos, CA (US)

Correspondence Address: COOPER & DUNHAM, LLP, 30 Rockefeller Plaza, 20th Floor, NEW YORK, NY, 10112, US

(73) Assignee: O2 MEDTECH, INC., Los Altos, CA (US)

(21) Appl. No.: 12/701,274

(22) Filed: Feb. 5, 2010

Related U.S. Application Data
(60) Provisional application No. 61/150,017, filed on Feb. 5, 2009.

(51) Int. Cl. A61B 5/1455

(52) U.S. Cl. 600/323

(57) ABSTRACT

Non-invasive spectrophotometric monitoring of oxygen saturation levels based on a combination of continuous wave spectrophotometry (CWS) and phase modulation spectrophotometry (PMS) is described. First information representative of absolute oxygen saturation levels in relatively shallow regions of a patient tissue volume are acquired from PMS-based monitoring thereof during a reference interval. Second information representative of non-absolute oxygen saturation levels in relatively deep regions of the tissue volume are acquired from CWS-based monitoring thereof during the reference interval. Based on the first and second information acquired during the reference interval, a mapping is automatically determined between the second information and estimated absolute oxygen saturation metrics for the relatively deep regions. On a continuing basis during a monitoring interval subsequent to the reference interval, the second information continuously acquired from CWS-based monitoring of the tissue volume are continuously mapped into estimated absolute oxygen saturation metrics, which are continuously displayed on a display output.

### CROSS-REFERENCE TO RELATED APPLICATIONS

This patent application claims the benefit of U.S. Provisional Ser. No. 61/150,017, filed Feb. 5, 2009, which is incorporated by reference herein.

### FIELD

This patent specification relates to the monitoring of a physiological condition of a patient using information from near-infrared (NIR) optical scans. More particularly, this patent specification relates to the monitoring of tissue oxygenation based on a combination continuous wave spectrophotometry (CWS) and phase-modulation spectrophotometry (PMS).

### BACKGROUND AND SUMMARY

The use of near-infrared (NIR) light as a basis for the measurement of biological properties or conditions in living tissue is particularly appealing because of its relative safety as compared, for example, to the use of ionizing radiation. Various techniques have been proposed for non-invasive NIR spectroscopy or NIR spectrophotometry (NIRS) of biological tissue. Generally speaking, these techniques are directed to detecting the concentrations of one or more chromophores in the biological tissue, such as blood hemoglobin in oxygenated (HbO) and deoxygenated (Hb) states.

As used herein, NIR tissue oxygenation level monitoring refers to the introduction of NIR radiation (e.g., in the 500-2000 nm range) into a tissue volume and the processing of received NIR radiation migrating outward from the tissue volume to generate at least one metric indicative of oxygenation level(s) in the tissue. One example of an oxygenation level metric is oxygen saturation, denoted herein by the symbol SO₂, which refers to the fraction or percentage of total hemoglobin in the tissue volume that is oxygenated hemoglobin. An NIR-based oxygen saturation reading can be classified as “absolute” or “non-absolute” in nature. An absolute SO₂ reading refers to an actual quantitative percentage of the total hemoglobin that is oxygenated hemoglobin for the tissue volume of interest. In contrast, a non-absolute SO₂ reading, which can alternatively be termed a “relative” or “trend-only” reading, refers to a measurement that cannot or should not be tied to such an actual quantitative percentage. By way of analogy, absolute SO₂ readings can be likened to an auto speedometer having a dial that is specifically printed with miles per hour or kilometers per hour numbers on it, whereas non-absolute SO₂ readings can be likened to an auto speedometer having a dial with no numbers printed on it, or that alternatively has an arbitrary scale of numbers printed on it.

NIR cerebral oxygenation level monitoring, which refers to the transcranial introduction of NIR radiation into the intracranial compartment and the processing of received NIR radiation migrating outward therefrom to generate at least one metric indicative of oxygenation level(s) in the brain, represents one particularly important type NIR tissue oxygenation level monitoring. One exemplary need for reliable determination of oxygen saturation levels in the human brain arises in the context of the millions of surgical procedures performed under general anesthesia every year. One statistic recited in U.S. Pat. No. 5,902,235 is that at least 2,000 patients die each year in the United States alone due to anesthetic accidents, while numerous other such incidents result in at least some amount of brain damage. Certain surgical procedures, particularly of a neurological, cardiac or vascular nature, may require induced low blood flow or pressure conditions, which inevitably involves the potential of insufficient oxygen delivery to the brain. Many surgical procedures also involve the possibility that a blood clot or other clottable material can break free, or otherwise get introduced into the bloodstream, and travel to the brain to cause a localized or widespread ischemic event therein. At the same time, the brain is highly intolerant to oxygen deprivation, and brain cells will die (become infarcted) within a few minutes if not sufficiently oxygenated. Accordingly, the availability of immediate, accurate and reliable information concerning brain oxygenation levels is of critical importance to anesthesiologists and surgeons, as well as other involved medical practitioners.

Pulse oximetry, in which infrared sources and detectors are placed across a thin part of the patient's anatomy such as a fingertip or earlobe, has arisen as a standard of care for all operating room procedures. However, pulse oximetry provides only a general measure of blood oxygenation as represented by the blood passing by the fingertip or earlobe sensor, and does not provide a measure of oxygen levels in vital organs such as the brain. In this sense, the surgeons in the operating room essentially “fly blind” with respect to brain oxygenation levels, which can be a major source of risk for patients (e.g., stroke) as well as a major source of cost and liability issues for hospitals and medical insurers.

Valid NIR cerebral oxygenation level readings can provide crucial monitoring data for the surgeon and other attending medical personnel, providing more direct data on brain oxygenation levels than pulse oximeters while being just as safe and non-invasive as pulse oximeters. Generally speaking, such systems involve the attachment of an NIR probe patch, or multiple such NIR probe patches, to the forehead and/or other available skin surface of the head. Each NIR probe patch usually comprises one or more NIR optical sources for introducing NIR radiation into the cerebral tissue and one or more NIR optical receivers for detecting NIR radiation that has migrated through at least a portion of the cerebral tissue. One or more oxygenation level metrics are then provided on a viewable display in a digital readout and/or graphical format.

FIG. 1A illustrates a conceptual block diagram of a continuous wave spectrophotometer (CWS) system 102 according to the prior art. CWS-based systems are known in the art and are discussed, for example, in WO1992/20273A2 and WO1996/16592A1. CWS system 102 comprises a CWS modulator 104 that modulates optical source(s) 106, the optical radiation propagating through tissue T to optical radiation detector(s) such as a photodiode 108. Electrical signals corresponding to the received optical radiation are demodulated by CWS demodulation circuitry 110 and processed by processor 112 to result in an output SO₂ reading 114 which, for conventional CWS-based systems. Generally speaking, as used herein, a CWS-based system is one for which intensity measurements, but no phase measurements, for the detected radiation are processed to compute SO₂ readings. At present, absolute measurement of chromophore concentration in CWS system is still not feasible due to difficulty in measuring optical pathlength of photons traversing the live tissue. Therefore, the pathlength of photons might be longer than the distance traveled between light source and detector. Hence, during a specific activity, only relative changes in chromophore concentration rather than absolute chromophore concentration can be calculated, by measuring the physiological range at a point of interest from a baseline level. Accordingly, the SO₂ reading 114 is denoted in FIG. 1 as a non-absolute (relative, trend-only) SO₂ metric.

FIG. 2 illustrates a conceptual block diagram of a phase modulation spectrophotometer (PMS) system 202 for providing oxygen saturation readings. PMS-based systems, which are sometimes termed intensity modulation spectroscopy systems and sometimes termed frequency domain spectroscopy systems, are known in the art and are discussed, for example, in U.S. Pat. No. 4,972,331, U.S. Pat. No. 5,187,672, and WO1994/21173A1. Generally speaking, as used herein, a PMS-based system is one for which both intensity measurements and phase measurements for the detected radiation are processed to compute SO₂ readings. PMS system 202 comprises a PMS modulator 204 that modulates optical source(s) 206, the optical radiation propagating through tissue T to an optical radiation detection system including collector optics 207 (for example, windows and prism reflectors in a probe patch) that transfer the optical radiation to optical fibers 208 that, in turn, transfer the optical radiation to a photomultiplier tube (PMT) 209. Electrical signals from the PMT tube 209 corresponding to the received optical radiation are demodulated by PMS demodulation circuitry 210 and processed by processor 212 to result in an output SO₂ reading 214 which, advantageously, can be an absolute oxygen saturation reading.

For oxygen saturation monitoring (SO₂ monitoring) in the brain it is often more desirable for to be provided with absolute SO₂ readings than relative SO₂ readings, for at least the reason that a given percentage drop in SO₂ level may, or may not, represent a critical ischemic situation. By way of example, it has been found in practice that absolute SO₂ readings in the range of 60%-80% are usually associated with non-problematic conditions, with the SO₂ reading varying within the 60%-80% range for any of a variety of normal, non-problematic reasons, whereas absolute SO₂ readings below 60% can be associated with a problematic ischemic condition. Accordingly, by way of example, a fifteen percent relative drop in SO₂ from an absolute reading of 75% to an absolute reading of 64%, as measured by a PMS-based system, can be considered non-problematic, while a fifteen percent relative drop in SO₂ from 65% to 55%, as measured by a PMS-based system, could be reason for alarm. However, if a CWS-based system is being used, the relative drop of fifteen percent is the only information being provided by the monitoring system, and therefore the medical personnel face an uncertain situation because they do not know if that drop is truly problematic or not, making relative SO₂ readings generally less desirable than absolute SO₂ readings in this environment.

Unfortunately, PMS-based systems contain certain practical limitations compared to CWS-based systems that make PMS-based system much more expensive and less robust in everyday clinical environments. Whereas CWS modulation rates are relatively low, typically only around 25 kHz or lower (not tending all the way to DC primarily to avoid unacceptable 1/f noise levels), PMS modulation rates are relatively very high in the 100 MHz-1000 MHz range. The lower modulation rate of CWS makes the modulation and demodulation circuitry relatively easy and less expensive to implement in comparison to PMS modulation and demodulation circuitry. Furthermore, electromagnetic interference issues become more important and complex in the PMS modulation range of 100 MHz 1000 MHz, for at least the reason that over-the-air television signals, FM radio signals, etc. fall in that frequency band, making electromagnetic shielding requirements more important and the performance of the device less robust.

Importantly, PMS-based systems further tend to suffer from a more limited penetration depth than CWS-based systems. Physically, in the relevant radiation wavelengths in the neighborhood of 700-800 nm, attenuation of propagating radiation is substantially higher when that radiation is modulated at 100 MHz-1000 MHz than when that radiation is modulated at only 25 KHz. Also, the detector size for PMS-based systems (see FIG. 2, A_{D,PMS}) needs to remain small in order for discernable signal phase delays to remain intact. Even when highly sensitive (and expensive, bulky, and complex) PMT detector systems are used, the source-to-detector spacing in PMS-based systems is more limited than for CWS-based systems. CWS-based systems are less sensitive to detector size, allowing larger-area detectors (see FIG. 1, A_{D,CWS}), and therefore greater source-to-detector spacing and/or the advantageous ability to use cheaper, less expensive detectors such as photodiodes rather than PMT detector systems. One thumbnail empirical relationship is that penetration depth tends to be about one-half of the source-detector spacing, for both CWS systems (D_{CWS}≈S_{CWS}/2, see FIG. 1) and PMS systems (D_{PMS}≈S_{PMS}/2, see FIG. 2). By way of nonlimiting numerical example, the source-detector spacing in many PMS-based systems is often limited to 4-6 cm, making the penetration depth limited to about 2-3 cm. In contrast, the source-detector spacing in many CWS-based systems can substantially greater than the range of 4-6 cm, although, as discussed supra, CWS-based systems such as those of FIG. 1 can only provide non-absolute, trend-only output readings.

FIGS. 3A-3E summarize, in simplified form, the well-accepted “slope method” that is applicable to PMS-based systems and, in a reduced form, to CWS-based systems. Descriptions of the slope method can be found, for example, in Fantini. Franceschini, and Gratton, “Semi-Infinite-Geometry Boundary Problem For Light Migration In Highly Scattering Media: A Frequency-Domain Study In The Diffusion Approximation,” J. Opt. Soc. Am. B, Vol. 11, pp. 2128-38 (1994) and Fantini, Hueber, and Franceschini, et. al., “Non-Invasive Optical Monitoring of the Newborn Piglet Brain Using Continuous-Wave and Frequency-Domain Spectroscopy,” Phys. Med. Biol., Vol. 44, pp. 1543-1563 (1999), each of which is incorporated by reference herein. For PMS-based systems, the basis of the slope method is (i) for any particular NIR radiation wavelength, a plot of log (r²I) versus r (where I is the measured intensity and r is the source-detector distance, FIG. 3B) has a relatively constant slope Kₐ over an appreciably useful range of distances, (ii) a plot of φ versus r (where φ is the measured phase, FIG. 3C) also has a relatively constant slope Kₚ over an appreciably useful range of distances, and (iii) the values of Kₐ and Kₚ can be used to compute the absorption coefficient μₐ and the effective or reduced scattering coefficient μₛ′ for that NIR radiation wavelength (FIG. 3D), where w is the angular frequency corresponding to the source intensity modulation and v is the speed of light in the tissue. For CWS-based systems, the same intensity-based slope Kₐ is computed (FIG. 3B), but there is no phase measurement available for a phase-based slope measurement. For CWS-based systems, the absorption coefficient μₐ is computed from the value of Kₐ in conjunction with a simple fixed estimate of the effective scattering coefficient μₛ′ (FIG. 3E).

For both PMS-based and CWS-based cases, the absorption coefficient μₐ for multiple NIR wavelengths (on opposite sides of the isosbestic wavelength for oxygenated and deoxygenated hemoglobin) can then be used to compute the oxygenated hemoglobin saturation value SO₂, such as by using the well-known empirical relationship of FIG. 3F for the particular NIR wavelengths of 690 nm and 830 nm. Generally speaking, consistent with the more precise measurement of the absorption coefficient μₐ based on both intensity and phase measurement, the SO₂ reading for the PMS-based case can be characterized as an absolute percentage value. Generally speaking, consistent with the generally rougher computation of the absorption coefficient μₐ, the SO₂ reading for the CW-based case measurements should be provided as a non-absolute (relative, trend-only) reading on an output display.

Thus, generally stated, the CWS-based system of FIG. 1 is capable of providing SO₂ readings applicable to substantially greater tissue depths than the PMS-based system of FIG. 2, but only in a non-absolute SO₂ context, while the PMS-based system of FIG. 2 is capable of providing absolute SO₂ readings, but only for generally shallower tissue depths than the CWS-based system of FIG. 1. It would be desirable to provide a non-invasive spectrophotometric monitoring system that is capable of providing absolute oxygen saturation level measurements applicable to relatively deep levels in the human brain, the measurements being sufficiently practical to obtain and yet being sufficiently reliable for use in surgical environments or other clinical settings in which the patient may slip from a non-ischemic condition to an ischemic condition. However, it is to be appreciated that the scope of the preferred embodiments described hereinbelow is not limited to cerebral oxygen saturation monitoring, but also includes devices and related methods for practical, reliable determination of absolute oxygen saturation levels in relatively deep parts of anatomy other than the human brain, such as the human kidney. Other issues arise as would be apparent to a person skilled in the art in view of the present disclosure.

According to one preferred embodiment, a method for non-invasive spectrophotometric monitoring of oxygen saturation levels based on a combination of combined continuous wave spectrophotometry (CWS) and phase modulation spectrophotometry (PMS) is provided. The method is applied for a patient monitoring session that includes (i) a reference interval, and (ii) a monitoring interval subsequent to the reference interval. First information acquired from PMS-based monitoring of a patient tissue volume during the reference interval is received, the first information being representative of one or more absolute oxygen saturation levels in one or more respective relatively shallow regions of the tissue volume. Second information acquired from CWS-based monitoring of the tissue volume during the reference interval is also received, the second information being representative of one or more non-absolute oxygen saturation levels in one or more respective relatively deep regions of the tissue volume. Based on the first and second information associated with the reference interval, a mapping is automatically determined between the second information and at least one estimated absolute oxygen saturation metric applicable to one or more respective relatively deep regions. Then, on a continuing basis during the monitoring interval, the second information acquired from the CWS-based monitoring is mapped into estimated absolute oxygen saturation metrics applicable to the one or more respective relatively deep regions by applying the determined mapping, and the estimated absolute oxygen saturation metrics are continuously displayed on a display output. In another preferred embodiment a computer readable medium tangibly embodying computer code is provided, the computer code causing all or a substantial part of the above-described method to be carried out when executed by one or more processors.

Also provided is a system for non-invasive spectrophotometric monitoring of oxygen saturation levels in a tissue volume of a patient during a patient monitoring session, the patient monitoring session including a reference interval and a monitoring interval subsequent to the reference interval. The system comprises a PMS subsystem for PMS-based monitoring of the tissue volume, the PMS subsystem generating first information representative of one or more absolute oxygen saturation levels in one or more respective relatively shallow regions of the tissue volume. The system further comprises a CWS subsystem for CWS-based monitoring of the tissue volume, the CWS subsystem generating second information representative of one or more non-absolute oxygen saturation levels in one or more respective relatively deep regions of the tissue volume. The system further comprises a processing system, such as a programmable computer, that is programmed to determine, based on the first information and the second information as acquired during the reference interval, a mapping between the second information and one or more estimated absolute oxygen saturation metrics applicable to the one or more relatively deep regions of the tissue volume. The programmable computer is further programmed to compute, on a continuing basis during the monitoring interval, the one or more estimated absolute oxygen saturation metrics applicable to the respective one or more relatively deep regions by applying the determined mapping to the second information as acquired during the monitoring interval. The system further comprises an output display for displaying, on a continuing basis during the monitoring interval, the one or more estimated absolute oxygen saturation metrics applicable to the respective one or more relatively deep regions of the tissue volume.

### BRIEF DESCRIPTION OF THE DRAWINGS

FIG. 1 illustrates tissue oxygen saturation monitoring using continuous wave spectrophotometry (CWS) according to the prior art;

FIG. 2 illustrate tissue oxygen saturation monitoring using phase modulation spectrophotometry (PMS) according to the prior art;

FIGS. 3A-3F illustrate a slope method for computing oxygen saturation levels;

FIG. 4 illustrates a hybrid CWS-PMS oxygen saturation monitoring system that uses combined continuous wave spectrophotometry (CWS) and phase modulation spectrophotometry (PMS) according to a preferred embodiment;

FIG. 5 illustrates oxygen saturation monitoring using a combination of continuous wave spectrophotometry (CWS) and phase modulation spectrophotometry (PMS) according to a preferred embodiment;

FIGS. 6A-6C illustrates a probe unit of a hybrid CWS-PMS cerebral oxygen level measurement system according to a preferred embodiment;

FIG. 7A illustrates a conceptual top view of the a probe unit of FIGS. 6A-6C as applied to the head of a patient;

FIG. 7B illustrates an output display of a hybrid CWS-PMS cerebral oxygen level measurement system hybrid according to a preferred embodiment;

FIG. 8A illustrates a conceptual top view of the a probe unit of FIGS. 6A-6C as applied to the head of a patient;

FIG. 8B illustrates an output display of a hybrid CWS-PMS cerebral oxygen level measurement system hybrid according to a preferred embodiment;

FIG. 9 illustrates a probe unit of a hybrid CWS-PMS renal oxygen level measurement system according to a preferred embodiment;

FIG. 10 illustrates a cross-section of an abdominal tissue volume to which is two of the probe units of FIG. 9;

FIGS. 11A-11B illustrate a user display of a hybrid CWS-PMS renal oxygen level measurement system according to a preferred embodiment; and

FIG. 12 illustrates a conceptual plot of source power for different CWS sources of a cerebral oxygen level measurement system according to a preferred embodiment.

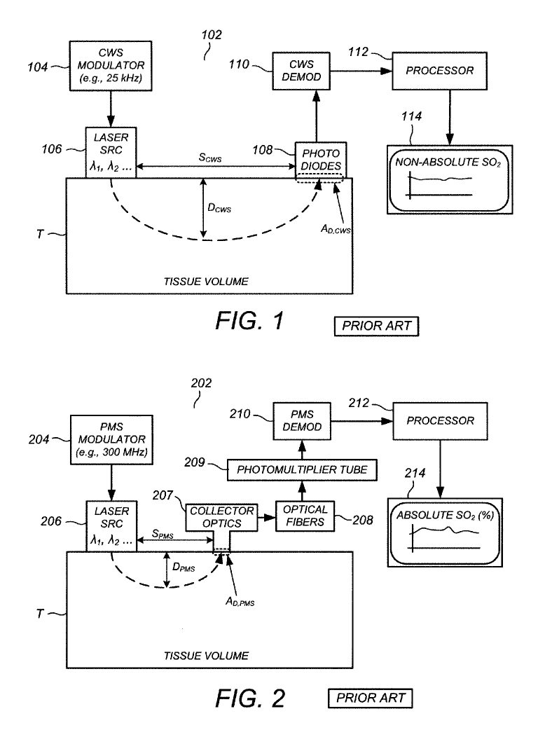

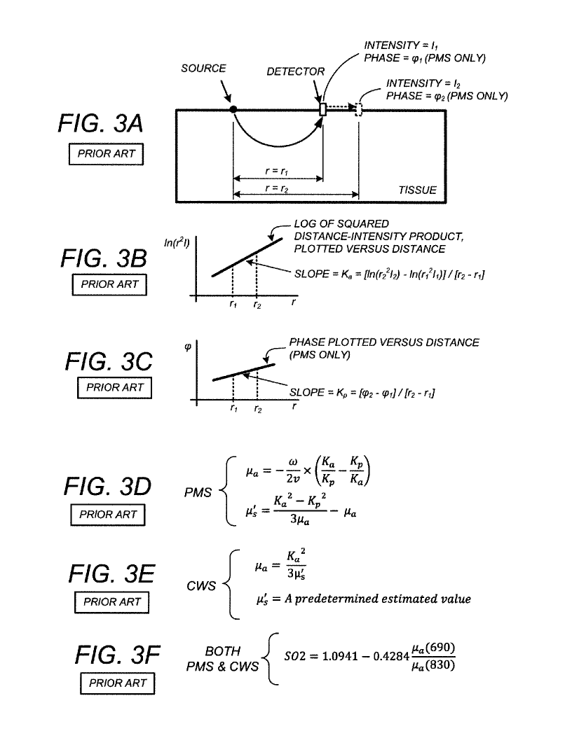

### DETAILED DESCRIPTION

Hybrid CWS-PMS cerebral oxygen saturation monitoring system using combined continuous wave spectrophotometry (CWS) and phase modulation spectrophotometry (PMS) according to one or more preferred embodiments is based at least in part on a finding that, for many practical clinical applications, it is sufficiently accurate and practical to assume that the SO₂ levels throughout the brain are substantially uniform prior to the beginning of a surgical procedure, the ingestion of a drug, the application of an external stimulus, or more generally some event (termed herein a “subject medical event”) over the course of which SO₂ monitoring will be desired. Thus, during a generally quiescent period subsequent to the mounting of the CWS and PMS hardware on the head of the patient but prior to the onset of the subject medical event, absolute SO₂ readings from the PMS hardware, which are technically limited in applicability to relatively shallow brain regions near the PMS source-detector pairs, can be considered as being applicable to all regions of the brain, including relatively deep-level regions that are technically only being “reached” by the CWS source-detector pairs. Based on this premise, absolute PMS-based SO₂ readings and non-absolute CWS SO₂ readings acquired during that quiescent period (termed herein a “reference interval”) can be processed to generate a mapping (which can be a direct scaling in a simplest preferred embodiment) between the non-absolute CWS SO₂ readings and an estimate of absolute SO₂ levels in the corresponding relatively deep regions of the brain. Once this mapping is determined, it can be applied on an ongoing basis subsequent to the onset of the medical event (during a “monitoring interval”) to compute estimated absolute SO₂ readings applicable to the relatively deep-level regions from the non-absolute CWS SO₂ readings.

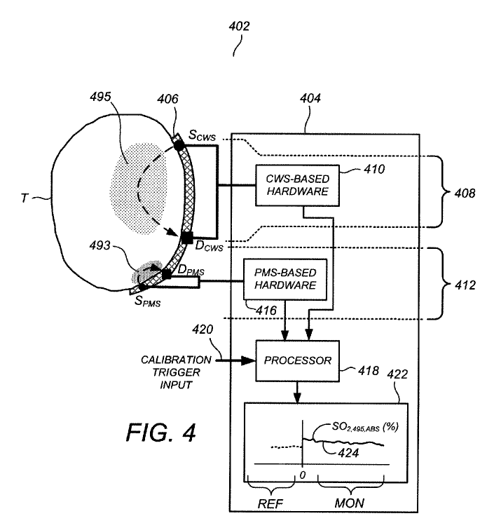

FIG. 4 illustrates a hybrid CWS-PMS oxygen saturation monitoring system 402 that uses combined continuous wave spectrophotometry (CWS) and phase modulation spectrophotometry (PMS) according to a preferred embodiment, comprising a housing 404 and a probe unit 406. The system 402 includes a CWS-based monitoring subsystem 408 comprising CWS-based hardware 410 and at least one CWS-based source-detector pair (S_{CWS}, C_{CWS}). The system 402 further includes a PMS-based monitoring subsystem 412 comprising PMS-based hardware 416 and at least one PMS source-detector unit (S_{PMS}, C_{PMS}). The system 402 further comprises a processor 418, an output display 422, the processor being configured and programmed to achieve the functionalities described herein. A user interface is provided that includes a calibration trigger input 420 that is manually instantiated by a user of the system (for example, just prior to the beginning of the subject medical event) to signal an end of a quiescent reference interval and the beginning of a monitoring interval. The calibration trigger input 420 can be provided in a variety of ways, such as with a hardware button, a softbutton pressable by mouse click, a touchscreen button, etc. An arbitrary time value “0” is shown in FIG. 4 as representing the time of the manual calibration trigger input from the user. Illustrated on the output display 422 is a time plot identifying a reference interval (REF) and a monitoring interval (MON), and displaying a time plot 424 of the desired estimated absolute SO₂ reading applicable to the relatively deep-level region 495 during the monitoring interval MON.

Conceptually illustrated in FIG. 4 is a relatively deep region 495 that is “reached” only by the CWS-based monitoring subsystem 408, and a relatively shallow region 493 to which the “reach” of the PMS-based monitoring subsystem 412. by the CWS-based monitoring subsystem 408. The spatial probe arrangements can be provided in a variety of different ways that cause the regions 495 and 493 to be spatially distinct, partially overlapping, or substantially overlapping, each without departing from the scope of the preferred embodiments. For one preferred embodiment, the source-detector spacing for the PMS source-detector pair units is less than about 6 cm, which corresponds to a thumbnail estimate of the relatively shallow region 493 as being less than about 3 cm deep, while the source-detector spacing for the CWS source-detector pair is greater than about 6 cm, which corresponds to a thumbnail estimate of the relatively deep region 495 as being greater than about 6 cm deep. Although this 3 cm depth demarcation (6 cm source-detector spacing demarcation) between “relatively shallow” and “relatively deep” has been found to be a useful demarcation for many of today's practical PMS and CWS systems, this example is by no means intended to limit the scope of the preferred embodiments. More generally, for purposes of the described preferred embodiments, the demarcation between “relatively shallow” and “relatively deep” depths can be associated with a practical maximum source-detector spacing reliably achieved by the PMS subsystem to be used, and which is exceeded by the CWS subsystem to be used. Thus, for example, if the particular PMS subsystem to be used has a reliably achieved practical maximum source-detector spacing of about 4 cm, then the demarcation between “relatively shallow” and “relatively deep” would be about 2 cm, whereas if the particular PMS subsystem to be used has a reliably achieved practical maximum source-detector spacing of about 8 cm, then the demarcation between “relatively shallow” and “relatively deep” would be about 4 cm.

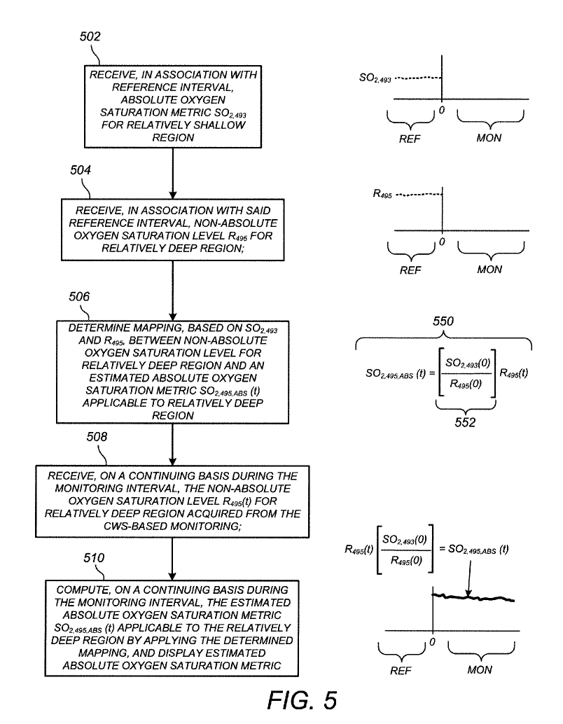

FIG. 5 illustrates steps executed by the processor 418 in conjunction with the user interface and user display 422 according to a preferred embodiment. At step 502, in association with the reference interval, an absolute oxygen saturation metric SO_{2,493} applicable to the relatively shallow region 493 (or other information from which that value can be derived) is received from the from the PMS monitoring subsystem 412. At step 504, in association with the reference interval, a non-absolute oxygen saturation metric R₄₉₅ applicable to the relatively deep region 495 (or other information from which that value can be derived) is received from the from the CWS monitoring subsystem 408. At step 506 a mapping is determined based on SO_{2,493} and R₄₉₅, between the non-absolute oxygen saturation level and an estimated absolute oxygen saturation metric SO_{2,495,ABS} (t) applicable to the relatively deep region 495. As one of many examples within the scope of the present teachings, FIG. 5 illustrates a relatively simple mapping 550 in which is a scaling of R₄₉₅ by a constant scaling factor 552, wherein the constant scaling factor 552 is that which, when multiplied by R₄₉₅(0) results in SO_{2,493}(0). The values for R₄₉₅(0) and SO_{2,493}(0) can be instantaneous values at time 0, or alternatively can be averaged over some or all of the reference interval. At step 508, on a continuing basis during the monitoring interval, the non-absolute oxygen saturation level R₄₉₅(t) for the relatively deep region 495 is received from the CWS-based monitoring subsystem 408. At step 510, on a continuing basis during the monitoring interval, the estimated absolute oxygen saturation metric SO_{2,495,ABS}(t) applicable to the relatively deep region 495 is computed by applying the determined mapping 550 to the non-absolute oxygen saturation level R₄₉₅(t) for the relatively deep region 495.

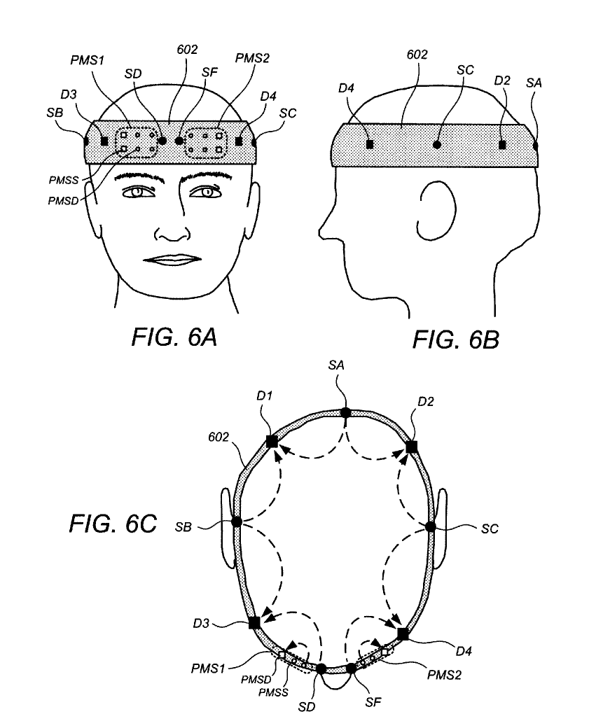

FIGS. 6A-6C illustrates a probe unit 602 of a hybrid CWS-PMS cerebral oxygen level measurement system according to a preferred embodiment, which represents an extension of the preferred embodiments of FIGS. 4-5 for the case of multiple PMS source-detector pair units (and therefore multiple relatively shallow regions of the tissue volume), multiple CWS sources, and multiple CWS detectors (and therefore multiple relatively deep regions of the tissue volume). Probe unit 602 comprises a headband or other means for supporting/mounting (i) a plurality of PMS source-detector units PMS1 and PMS2, each including plural sources PMSS and detectors PMSD, (ii) a plurality of CWS sources SA, SB, SC, SD, and SF, and (iii) a plurality of CWS detectors D1, D2, D3, and D4 to the skin of the head of the patient around its periphery in a region above the ears and eyebrows, as shown. Preferably, the head is shaved so that good optical coupling can be achieved all around the head, although it is not outside the scope of the preferred embodiments for “hairbrush” style fiber couplings to be used to obviate the need for shaving the head.

While many components of the probe unit 602 are omitted from the drawings for clarity of presentation (for example, fiber couplings, optical shielding, waveguides, etc.), it is to be appreciated that a person skilled in the art would be able to construct a probe unit and associated system according to the preferred embodiments in view of the present disclosure without undue experimentation. Unless indicated otherwise herein, any particular PMS source-detector unit PMS1, PMS2, etc., referenced herein shall be presumed to be accompanied by the necessary radiation collection optics, optical fibers, PMT tube(s), PMS demodulator circuitry, PMS signal processing circuitry, and output display devices as necessary to implement an overall PMS cerebral oxygen level measurement unit that provides a corresponding absolute SO₂ reading.

The plurality of CWS sources and detectors form the following individual source-detector pairs: SA-D1, SB-D1, SB-D3, SD-D3, SF-D4, SC-D4, SC-D2, and SA-D2. According to a preferred embodiment, in order to increase CWS source-detector distance and thereby increase CWS penetration depth, each of the CWS detectors comprises a photomultiplier tube (PMT)-based radiation detection scheme. However, provided that sufficient source-detector spacing is facilitated, it would not be outside the scope of the present teachings for photodiode-based detection schemes to be used. Unless indicated otherwise herein, any particular CWS source-detector pair referenced herein shall be presumed to be accompanied by the necessary radiation collection optics, optical fibers, PMT tube(s), CWS demodulator circuitry, and CWS signal processing circuitry as necessary to generate a corresponding relative SO2 reading. According to a preferred embodiment, this relative SO₂ reading is further processed, as described hereinbelow, such that a clinically meaningful absolute SO₂ reading is provided that corresponds to that CWS source-detector pair.

In operation, only one PMS source or CWS source is firing at any particular moment in time, and is firing at only one of its two or more source wavelengths (e.g., 690 nm or 830 nm). Because the NIR optical signal loss in living tissue such as the brain is extraordinarily high (about a factor of 10 for every cm of source-detector distance), CWS measurement pairs are only established for directly adjacent sources and detectors. However, it would not be outside the scope of the present teachings to also use non-adjacent CWS source-detector pairs (for example, the pair SA-D3) in the event that a meaningful reading could be acquired at D3 of a signal originating at the source SA.

In the preferred embodiment of FIGS. 6A-6C the CWS sources SD, SB, SA, SC, and SF can be characterized as being at “clockface coordinates” of about 12:30, 3:00, 6:00, 9:00 and 11:30, respectively, where the nose is considered to be at 12:00, while the CWS detectors D3, D1, D2, and D4 can be considered to be at about 1:30, 4:30, 7:30, and 10:30, respectively. According to another preferred embodiment (not shown), a plurality of CWS sources are distributed at 1:30, 4:30, 7:30, and 10:30 and a plurality of CWS detectors are distributed at 12:00, 3:00, 6:00, and 9:00.

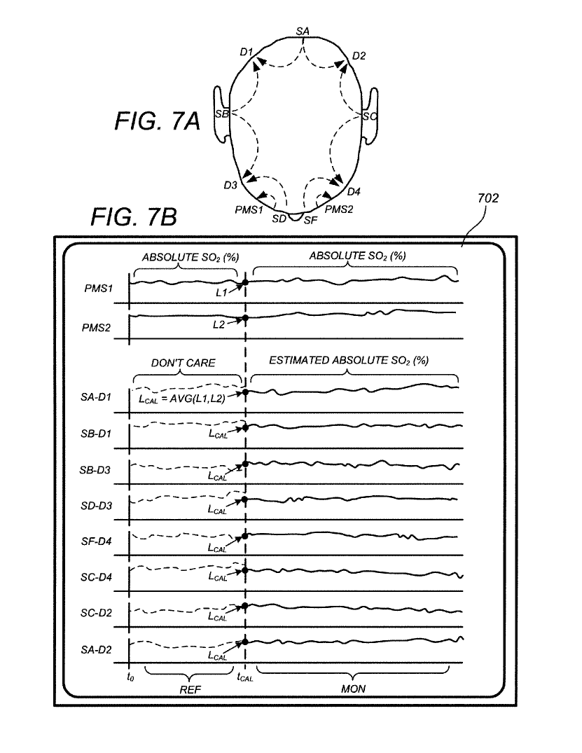

FIG. 7A illustrates a simplified version of FIG. 6C (omitting the headband and source/detector iconic shapes), and FIG. 7B illustrates an output display 702 according to a preferred embodiment, with annotations added for illustrating particular applications of the method of FIGS. 4-5 supra for the multiple deep-region, multiple shallow-region case. It has been found useful, practical, and sufficiently accurate to assume the head to have a substantially uniform SO₂ prior to the beginning of a surgical procedure, the ingestion of a drug, the application of an external stimulus, or more generally some event (termed herein a “subject medical event”) over the course of which SO₂ monitoring will be desired, and to calibrate one or more CWS source-detector pairs at some point in time t_{CAL} prior to the onset of the subject medical event based on absolute PMS-based SO₂ readings acquired by one or more PMS source-detector units at the time t_{CAL} that are located with or near the one or more CWS source-detector pairs. The calibration process comprises (i) computing an absolute PMS-based SO₂ reading L_{CAL} representative of the assumed-uniform tissue at the time t_{CAL}, such as by taking an average of the absolute PMS-based SO₂ readings of the one or more PMS source-detector units, (ii) for each CWS source-detector pair, determining a numerical calibration factor (scaling factor) that, when multiplied by the relative SO₂ reading at time t_{CAL}, would result in an absolute output reading of L_{CAL} for that CWS source-detector pair, and (iii) from time t_{CAL} onward, setting the absolute SO₂ reading for that CWS source-detector pair equal to the product of that numerical calibration factor and the relative SO₂ reading corresponding to that CWS source-detector pair. The time t_{CAL} should be a sufficient interval (probably about 1 minute or so depending on the system hardware and patient coupling equipment) after an initial connection or reset time t₀ to allow the absolute and relative readings to reach a reasonably quiescent state.

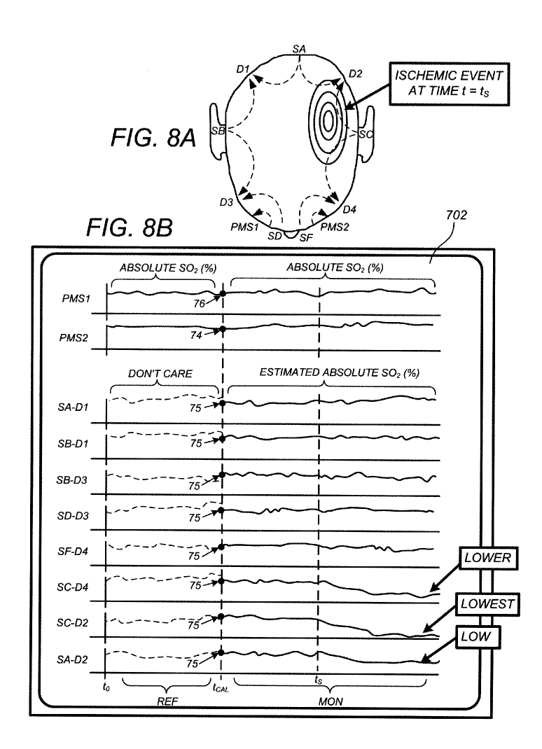

FIGS. 8A-8B illustrate an exemplary numerical example corresponding to the preferred embodiment of FIGS. 7A-7B, respectively, for an exemplary scenario in which an ischemic event begins to affect a part of the brain at a time tₛ during the subject medical event. At the time of calibration t_{CAL}, a reference PMS-based absolute SO₂ reading is computed by averaging the PMS-based absolute SO₂ readings for the two relatively shallow regions (e.g., 75% is the average of 76% and 74%), and then a distinct scaling factor is computed for each relatively deep tissue region such that, when multiplied by the non-absolute CWS-based SO₂ metric for that deep region at time t_{CAL}, results in the value of that reference PMS-based absolute SO₂ reading (e.g., that results in a value of 75%). Thereafter, those scaling factors are applied to the corresponding non-absolute CWS-based SO₂ metric for each deep region to result in the estimated absolute SO₂ reading applicable to each deep region. Advantageously, the medical professional can readily see a downward trend pattern in the graphical plots (or, in an alternative preferred embodiment, numerical output readings) that can be readily used to localize the area of the ischemic event. As a further advantage, the severity of ischemic event can be assessed by looking at the absolute SO₂ readings for the relevant CWS source-detector pairs, and seeing if they are falling below a dangerous absolute lower limit (such as 60% for the numerical clinical example given previously).

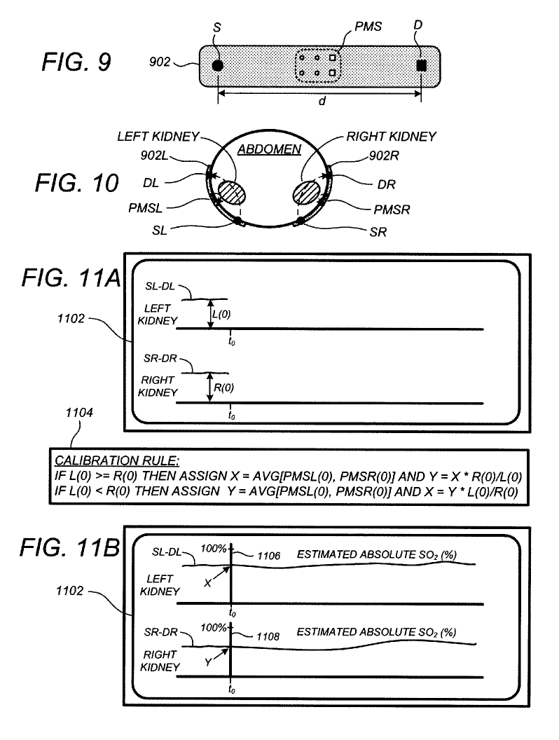

FIG. 9 illustrates a probe unit 902 of a hybrid CWS-PMS renal oxygen level measurement system according to a preferred embodiment, which is analogous to the probe unit 602 of FIGS. 6A-6C, supra, except that it comprises a single CWS source-detector pair and a single PMS measurement unit. As with the CWS source-detector pairs of FIGS. 6A-6C, it is preferable for a photomultiplier tube (PMT)-based detection system (not shown) to be used for optical detection, so that the distance “d” is between about 15-16 cm. For another preferred embodiment the distance “d” can be between 10-20 cm. A PMS source-detector unit “PMS” is provided approximately halfway between the source S and detector D.

FIG. 10 illustrates a cross-section of an abdomen to which is applied an instance of the probe unit 902 for each of the left kidney (unit 902L) and right kidney (unit 902R). While calibration of a hybrid CWS-PMS renal oxygen level measurement system is presented herein assuming dual simultaneous probe units 902L and 902R, the methods can be readily adapted for a single probe unit 902 that is shifted manually between the left and right kidneys. As illustrated conceptually in FIG. 10, the PMS units PMSL and PMSR provide absolute SO₂ for relatively limited depths into subdermal fat tissue, while the CWS source-detector pairs SL-DL and SR-DR achieve substantially greater penetration depth that can encompass a significant portion of the kidney.

FIGS. 11A-11B illustrate a user display 1102 of a hybrid CWS-PMS renal oxygen level measurement system according to a preferred embodiment at different times, with annotations added for illustrating a method for calibrating CWS source-detector pairs of a hybrid CWS-PMS renal oxygen level measurement system according to a preferred embodiment. Unlike with the brain oxygen saturation monitoring scenarios described above, it is substantially less likely that the monitoring system will have been put in place before the onset of an ischemic kidney event (or suspected ischemic kidney event). Rather, it will be more likely that the monitoring system will be used to detect whether an ischemic kidney event is already taking place, such as when the patient arrives at the medical facility with kidney pain, although the preferred embodiments can certainly be used for ongoing prospective monitoring of an asymptomatic patient as well.

It has been found useful, practical, and sufficiently accurate to assume that an (i) ischemic kidney event, if it has occurred, has only affected one kidney and not the other, and that (ii) the general area of the unaffected kidney including the tissue between that kidney and the probe unit 902 can be approximated as having a generally uniform SO₂ level. Shown in FIG. 11A are plots of the relative SO₂ readings for the left and right kidneys at some time subsequent to the placement of the monitoring system on the patient (FIG. 10) or system reset such that a quiescent state is reached (e.g., about 1 minute afterward), but prior to a calibration procedure according to a preferred embodiment, which can be instituted at an otherwise arbitrary time t₀. Absolute SO₂ readings are also being taken by the PMS units PMSL and PMSR and output on the user display but are omitted from FIGS. 11A-11B for clarity of presentation.

As of the time t₀, the absolute SO₂ readings from the PMS units PMSL and PMSR are presumed to have reached reasonably quiescent values denoted here as PMSL(0) and PMSR(0), respectively, or can be time-averaged to produce those values. As of the time t₀, the relative SO₂ readings from SL-DL and SR-DR are presumed to have reached reasonably quiescent values denoted as L(0) and R(0), respectively, or can be time averaged to produce those values. According to a preferred embodiment, a calibration rule (i.e., a mapping) is applied to generate an absolute SO₂ level X to which the SL-DL relative output is mapped by virtue of the scaling axis 1106, as well as to generate an absolute SO₂ level Y to which the SR-DR relative output is mapped by virtue of the scaling axis 1108, and these computed scalings remain fixed thereafter. According to one preferred embodiment, the calibration rule, as illustrated in box 1104, is that if L(0) is greater than or equal to R(0) (that is, the right-side kidney is detected as having the ischemic condition), then X is assigned to the average of PMSL(0) and PMSR(0) Y is assigned to the value of X times R(0)/L(0), whereas if L(0) is less than R(0) (that is, the left-side kidney is detected as having the ischemic condition), then Y is assigned to the average of PMSL(0) and PMSR(0) and X is assigned to the value of Y times L(0)/R(0).

According to another preferred embodiment, the calibration rule is that if L(0) is greater than or equal to R(0), then X is assigned to PMSL(0) and Y is assigned to the value of X times R(0)/L(0), whereas if L(0) is less than R(0), then Y is assigned to PMSR(0) and X is assigned to the value of Y times L(0)/R(0). In other words, the calibration to an absolute value is based on an SO₂ uniformity assumption with the nearby PMS reading for whichever kidney (left or right) is yielding the higher CWS relative SO₂ value, and then the opposing side is scaled to an absolute value based on a ratio of the lower CWS relative SO₂ value to the higher CWS relative SO₂ value.

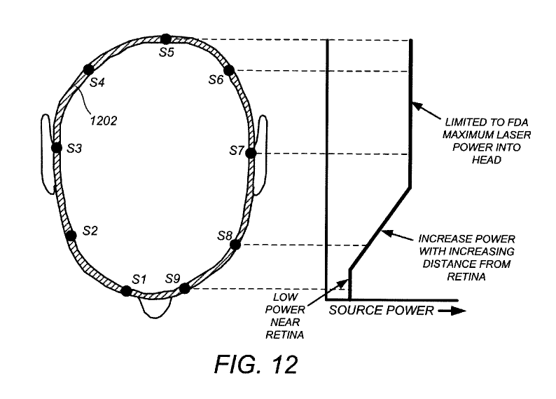

FIG. 12 illustrates a conceptual plot of source power for a probe unit 1202 of a cerebral oxygen level measurement system according to a preferred embodiment, which can optionally be a hybrid CWS-PMS probe unit, although the scope of the present teachings is not so limited. Detectors are omitted from FIG. 12 for clarity of presentation, with only sources being shown. According to a preferred embodiment, the average operating laser power for sources near the back of the head, which are very distant from the retina, is turned up very high and is limited only by FDA regulations on laser power to the head in general. In contrast, as the position of the source draws nearer to the front of the head, the source power is reduced in order to avoid retinal damage or unpleasant visual sensations due to laser light incident upon the retina. By maximizing power in this way, safety and patient comfort are accommodated, while also maximizing penetration depth for brain tissue closer to the back of the head, since source-detector separation can be increased with increased amounts of source power. By way of example and not by way of limitation, the average laser power for sources S1 and S9 may be limited to about 30 mW due to their proximity to the retina, whereas the average laser power for sources S3-S7 might be about 500 mW depending on applicable FDA regulatory limits, and keeping in mind that only one of them is firing at any given time.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, whereas one or more of the above-described preferred embodiments includes a hybrid CWS-PMS scheme in which absolute PMS SO₂ readings are used to provide a basis for calibrating relative CWS SO₂ readings to an absolute scale, in an alternative preferred embodiment there is provided a hybrid TRS (time resolved spectrophotometry)-PMS scheme in which absolute TRS SO₂ readings are used to provide a basis for calibrating non-absolute CWS SO₂ readings to an absolute scale. Therefore, reference to the details of the embodiments are not intended to limit their scope, which is limited only by the scope of the claims set forth below.

What is claimed is:

1. A method for non-invasive spectrophotometric monitoring of oxygen saturation levels in a tissue volume of a patient during a patient monitoring session, said patient monitoring session including a reference interval and a monitoring interval subsequent to said reference interval, comprising:
receiving, in association with said reference interval, first information acquired from phase modulation spectrophotometry-based (PMS-based) monitoring of the tissue volume, said first information being representative of at least one absolute oxygen saturation level in a respective at least one relatively shallow region of the tissue volume;
receiving, in association with said reference interval, second information acquired from continuous wave spectrophotometry-based (CWS-based) monitoring of the tissue volume, said second information being representative of at least one non-absolute oxygen saturation level in a respective at least one relatively deep region of the tissue volume;
determining, based on said first and second information associated with the reference interval, a mapping between said second information and at least one estimated absolute oxygen saturation metric applicable to the respective at least one relatively deep region of the tissue volume;
receiving, on a continuing basis during the monitoring interval, the second information acquired from the CWS-based monitoring of the tissue volume;
computing, on a continuing basis during the monitoring interval, the at least one estimated absolute oxygen saturation metric applicable to the respective at least one relatively deep region by applying said determined mapping to said second information received during the monitoring interval;
displaying, on a continuing basis during the monitoring interval, said at least one estimated absolute oxygen saturation metric applicable to the respective at least one relatively deep region on an output display.

2. The method of claim 1, the method further comprising:
providing a hybrid PMS-CWS monitoring unit including said output display, a CWS monitoring subsystem including at least one CWS source and at least one CWS detector, a PMS monitoring subsystem including at least one PMS source-detector unit, and a user interface capable of receiving a calibration trigger input from a user;
prior to said reference interval, coupling said at least one CWS source, said at least one CWS detector, and said at least one PMS source-detector unit to the surface of the tissue volume; and
at an end of said reference interval, manually providing the calibration trigger input to the user interface of the hybrid CWS-PMS monitoring unit to instantiate said mapping determination.

3. The method of claim 2, wherein said tissue volume corresponds to the head of the patient, wherein said reference interval is caused to occur during a assumed non-ischemic quiescent period in which cerebral oxygen saturation is more likely to be uniform throughout the head of the patient, and wherein said calibration trigger input is caused to occur prior to instantiation of a medical event during which anomalous conditions may cause ischemic cerebral conditions to occur, whereby said output display of said least one estimated absolute oxygen saturation metric facilitates detection of such cerebral ischemic conditions in deep brain tissue.

4. The method of claim 3, said at least one CWS source and said at least one CWS detector establishing at least one CWS source-detector pair, each CWS source-detector pair corresponding to one of the at least one relatively deep regions and having a source-detector spacing greater than about 6 cm, each PMS source-detector unit corresponding to one of the at least one relatively shallow regions and having a source-detector spacing of less than about 6 cm.

5. The method of claim 4, wherein said mapping determination comprises:
processing said second information associated with said reference interval to generate a reference CWS-based non-absolute oxygen saturation metric for each said at least one relatively deep region;
processing said first information associated with said reference interval to generate a reference PMS-based absolute oxygen saturation metric; and
for each said at least one relatively deep region, computing a fixed scaling factor that, when multiplied by said reference CWS-based non-absolute oxygen saturation metric, results in said reference PMS-based absolute oxygen saturation metric;
and wherein said computing on the continuous basis during the monitoring interval comprises (i) processing the second information acquired during the monitoring interval to generate a current CWS-based non-absolute oxygen saturation metric for each said at least one relatively deep region, and (ii) scaling the current CWS-based non-absolute oxygen saturation metric for each relatively deep region by the fixed scaling factor for that relatively deep region to generate the estimated absolute oxygen saturation metric applicable to that relatively deep region.

6. The method of claim 5, wherein a plurality of said PMS source-detector units are coupled to the surface of the head, and wherein said processing said first information associated with said reference interval to generate the reference PMS-based absolute oxygen saturation metric comprises:
generating a separate PMS-based absolute oxygen saturation metric for the relatively shallow region corresponding to each of the at least one PMS source-detector units; and
computing said reference PMS-based absolute oxygen saturation metric as an average of said separate PMS-based absolute oxygen saturation metrics.

7. The method of claim 5, wherein a plurality of said CWS sources are coupled to the head surface including a first plurality of CWS sources positioned farther than a predetermined threshold distance from a retina of the patient and a second plurality of CWS sources positioned nearer than said predetermined threshold distance from the retina, wherein said first plurality of CWS sources are operated at a maximum source power for the human head according to regulatory guidelines, and wherein said second plurality of CWS sources are operated at source powers that decrease with decreasing distance to the retina.

8. The method of claim 5, wherein a plurality of said CWS source-detector pairs are established around the head corresponding a respective plurality of the relatively deep regions, and wherein said output display includes a separate graphical trace for each of the corresponding estimated absolute oxygen saturation metrics, whereby localization of ischemic conditions in the deep brain tissue during the medical event is facilitated.

9. The method of claim 2, wherein said tissue volume includes both kidneys of the patient, and wherein, for each kidney, a CWS source-detector pair and a PMS source-detector pair are coupled to the surface of the tissue volume near that kidney, said CWS source-detector pair having a source-detector spacing of at least two times a depth of the kidney beneath the tissue volume surface.

10. The method of claim 9, said reference interval being caused to occur during an assumed single-kidney ischemic event, said calibration trigger input being caused to occur prior to treatment thereof or recovery therefrom, wherein said mapping determination comprises:
processing said second information associated with said reference interval to generate a reference CWS-based non-absolute oxygen saturation metric for each said kidney;
identifying one kidney as ischemic and the other kidney as non-ischemic by comparison of said reference CWS-based non-absolute oxygen saturation metrics;
processing said first information associated with said reference interval to generate a reference PMS-based absolute oxygen saturation metric, wherein said reference PMS-based absolute oxygen saturation metric is assigned to one of (i) a PMS-based oxygen saturation metric corresponding to the PMS source-detector pair nearer the non-ischemic kidney, and (ii) an average of the PMS-based oxygen saturation metrics for the PMS source-detector pairs;
computing a first fixed scaling factor that, when multiplied by the reference CWS-based non-absolute oxygen saturation metric for the non-ischemic kidney, results in said reference PMS-based absolute oxygen saturation metric; and
computing a second fixed scaling factor equal to the first scaling factor times a ratio of the CWS-based non-absolute oxygen saturation metric for the ischemic kidney to the CWS-based non-absolute oxygen saturation metric for the non-ischemic kidney;
and wherein, for a duration of said monitoring interval subsequent to said reference interval, said mapping comprises (i) for the non-ischemic kidney, scaling the corresponding CWS-based non-absolute oxygen saturation metric by said first fixed scaling factor to generate the estimated absolute oxygen saturation metric applicable thereto, and (ii) for the ischemic kidney, scaling the corresponding CWS-based non-absolute oxygen saturation metric by said second fixed scaling factor to generate the estimated absolute oxygen saturation metric applicable thereto.

11. The method of claim 1, wherein optical radiation within a wavelength range of 600 nm-1400 nm is used for both said CWS-based monitoring and PMS-based monitoring of the tissue volume.

12. The method of claim 1, wherein optical detection for both said CWS-based monitoring and PMS-based monitoring of the tissue volume is performed using photomultiplier tubes (PMTs).

13. A system for non-invasive spectrophotometric monitoring of oxygen saturation levels in a tissue volume of a patient during a patient monitoring session, the patient monitoring session including a reference interval and a monitoring interval subsequent to the reference interval, comprising:
a phase modulation spectrophotometry (PMS) subsystem for PMS-based monitoring of the tissue volume, the PMS subsystem generating first information representative of at least one absolute oxygen saturation level in a respective at least one relatively shallow region of the tissue volume;
a continuous wave spectrophotometry (CWS) subsystem for CWS-based monitoring of the tissue volume, the CWS subsystem generating second information representative of at least one non-absolute oxygen saturation level in a respective at least one relatively deep region of the tissue volume;
a computer coupled with said PMS subsystem and said CWS subsystem and being programmed to: (a) determine, based on said first information and said second information as acquired during said reference interval, a mapping between said second information and at least one estimated absolute oxygen saturation metric applicable to the respective at least one relatively deep region of the tissue volume; and (b) compute, on a continuing basis during the monitoring interval, the at least one estimated absolute oxygen saturation metric applicable to the respective at least one relatively deep region by applying said determined mapping to said second information as acquired during the monitoring interval; and
an output display for displaying, on a continuing basis during the monitoring interval, the at least one estimated absolute oxygen saturation metric applicable to the respective at least one relatively deep region of the tissue volume.

14. The system of claim 13, further comprising a user interface configured to receive a calibration trigger input from a user, the calibration trigger input providing a time point that separates the reference interval from the monitoring interval and causing said computer to instantiate said determination of said mapping.

15. The system of claim 14, wherein said determination of said mapping comprises:
processing said second information acquired during said reference interval to generate a reference CWS-based non-absolute oxygen saturation metric for each said at least one relatively deep region;
processing said first information acquired during said reference interval to generate a reference PMS-based absolute oxygen saturation metric; and
for each said at least one relatively deep region, computing a fixed scaling factor that, when multiplied by said reference CWS-based non-absolute oxygen saturation metric, results in said reference PMS-based absolute oxygen saturation metric;
and wherein said computing on the continuous basis during the monitoring interval comprises (i) processing the second information acquired during the monitoring interval to generate a current CWS-based non-absolute oxygen saturation metric for each said at least one relatively deep region, and (ii) scaling the current CWS-based non-absolute oxygen saturation metric for each relatively deep region by the fixed scaling factor for that relatively deep region to generate the estimated absolute oxygen saturation metric applicable to that relatively deep region.

16. The system of claim 15, wherein said tissue volume corresponds to the head of the patient, wherein said PMS subsystem comprises at least one PMS source-detector pair unit for coupling to the head of the patient, the PMS source-detector pair unit having a source-detector spacing less than about 6 cm, and wherein said CWS subsystem comprises a plurality of CWS sources and a plurality of CWS detectors for coupling to the head of the patient, the CWS sources and CWS detectors establishing a plurality of CWS source-detector pairs, each CWS source-detector pair corresponding to one of the at least one relatively deep regions and having a source-detector spacing greater than about 6 cm.

17. The system of claim 16, said CWS subsystem and said PMS subsystem each use optical radiation within a wavelength range of 600 nm-1400 nm, and wherein each said CWS subsystem and PMS subsystem comprises photomultiplier tubes (PMTs) for performing optical detection.

18. A computer readable medium tangibly embodying one or more sequences of instructions wherein execution of the one or more sequences of instructions by one or more processors causes the one or more processors to facilitate non-invasive spectrophotometric monitoring of oxygen saturation levels in a tissue volume of a patient during a patient monitoring session, said patient monitoring session including a reference interval and a monitoring interval subsequent to said reference interval, including performing the steps of:
receiving, in association with said reference interval, first information acquired from phase modulation spectrophotometry-based (PMS-based) monitoring of the tissue volume, said first information being representative of at least one absolute oxygen saturation level in a respective at least one relatively shallow region of the tissue volume;
receiving, in association with said reference interval, second information acquired from continuous wave spectrophotometry-based (CWS-based) monitoring of the tissue volume, said second information being representative of at least one non-absolute oxygen saturation level in a respective at least one relatively deep region of the tissue volume;
determining, based on said first and second information associated with the reference interval, a mapping between said second information and at least one estimated absolute oxygen saturation metric applicable to the respective at least one relatively deep region of the tissue volume;
receiving, on a continuing basis during the monitoring interval, the second information acquired from the CWS-based monitoring of the tissue volume;
computing, on a continuing basis during the monitoring interval, the at least one estimated absolute oxygen saturation metric applicable to the respective at least one relatively deep region by applying said determined mapping to said second information received during the monitoring interval;
causing to be displayed, on a continuing basis during the monitoring interval, said at least one estimated absolute oxygen saturation metric applicable to the respective at least one relatively deep region on an output display.

19. The computer readable medium of claim 18, wherein said mapping determination comprises:
processing said second information associated with said reference interval to generate a reference CWS-based non-absolute oxygen saturation metric for each said at least one relatively deep region;
processing said first information associated with said reference interval to generate a reference PMS-based absolute oxygen saturation metric; and
for each said at least one relatively deep region, computing a fixed scaling factor that, when multiplied by said reference CWS-based non-absolute oxygen saturation metric, results in said reference PMS-based absolute oxygen saturation metric;
and wherein said computing on the continuous basis during the monitoring interval comprises (i) processing the second information acquired during the monitoring interval to generate a current CWS-based non-absolute oxygen saturation metric for each said at least one relatively deep region, and (ii) scaling the current CWS-based non-absolute oxygen saturation metric for each relatively deep region by the fixed scaling factor for that relatively deep region to generate the estimated absolute oxygen saturation metric applicable to that relatively deep region.

20. The computer readable medium of claim 18, wherein said processing said first information associated with said reference interval to generate the reference PMS-based absolute oxygen saturation metric comprises:
computing from said first information a plurality of local PMS-based absolute oxygen saturation metric corresponding to different relatively shallow regions of the tissue volume; and
computing said reference PMS-based absolute oxygen saturation metric as an average of said local PMS-based absolute oxygen saturation metrics.
